(19) United States
(12) Patent Application Publication
(10) Pub. No.: US 2016/0008556 A1
(43) Pub. Date: Jan. 14, 2016

(54) NEEDLE INSERTION SYSTEM AND METHOD FOR INSERTING A MOVABLE NEEDLE INTO A VERTEBRATE SUBJECT

(71) Applicant: Elwha LLC, Bellevue, WA (US)

(72) Inventors: Michael H. Baym, Cambridge, MA (US); Philip A. Eckhoff, Kirkland, WA (US); Roderick A. Hyde, Redmond, WA (US); Jordin T. Kare, Seattle, WA (US); Gary L. McKnight, Bothell, WA (US); Tony S. Pan, Bellevue, WA (US); Lowell L. Wood, JR., Bellevue, WA (US)

(21) Appl. No.: 14/328,444

(22) Filed: Jul. 10, 2014

(51) Int. Cl. A61M 5/46

(52) U.S. Cl. CPC A61M 5/46

(57) ABSTRACT

A needle insertion system is disclosed that includes a moveable hollow needle having a distal tip configured to be transdermally inserted into a vertebrate subject at an insertion-target region; at least one sensor operable to detect skin structure at a position in proximity to the distal tip of the moveable needle in the insertion-target region; and control circuitry operably coupled to the at least one sensor and configured to receive information therefrom indicating the skin structure at the insertion-target region in proximity to the distal tip, and the control circuitry configured to output moveable needle targeting instructions to control movement of the moveable needle into the skin in response to one or more signals from the at least one sensor detecting the skin structure in proximity to the distal tip. A method for inserting a movable needle of a needle insertion system into a vertebrate subject is disclosed.

If an Application Data Sheet (ADS) has been filed on the filing date of this application, it is incorporated by reference herein. Any applications claimed on the ADS for priority under 35 U.S.C. §§119, 120, 121, or 365(c), and any and all parent, grandparent, great-grandparent, etc. applications of such applications, are also incorporated by reference, including any priority claims made in those applications and any material incorporated by reference, to the extent such subject matter is not inconsistent herewith.

### CROSS-REFERENCE TO RELATED APPLICATIONS

The present application claims the benefit of the earliest available effective filing date(s) from the following listed application(s) (the “Priority Applications”), if any, listed below (e.g., claims earliest available priority dates for other than provisional patent applications or claims benefits under 35 USC §119(e) for provisional patent applications, for any and all parent, grandparent, great-grandparent, etc. applications of the Priority Application(s)).

Priority Applications:

None.

If the listings of applications provided above are inconsistent with the listings provided via an ADS, it is the intent of the Applicant to claim priority to each application that appears in the Domestic Benefit/National Stage Information section of the ADS and to each application that appears in the Priority Applications section of this application.

All subject matter of the Priority Applications and of any and all applications related to the Priority Applications by priority claims (directly or indirectly), including any priority claims made and subject matter incorporated by reference therein as of the filing date of the instant application, is incorporated herein by reference to the extent such subject matter is not inconsistent herewith.

### SUMMARY

A needle insertion system and a method for inserting a movable hollow needle of a needle insertion system transdermally into a vertebrate subject are disclosed herein. The needle insertion system and the method include one or more sensors for determining skin structure at a position in proximity to the distal tip of the moveable needle at an insertion-target region on the skin of a vertebrate subject and for locating a position for a moveable needle for injection of a medicament. The needle insertion system and the method include control circuitry to move the needle to a defined depth based on the sensed skin structure at the insertion-target region in proximity to the distal tip of the moveable hollow needle prior to injecting the medicament into the vertebrate subject. A needle insertion system is disclosed that includes a moveable hollow needle having a distal tip configured to be transdermally inserted into a vertebrate subject at an insertion-target region; at least one sensor operable to detect skin structure at a position in proximity to the distal tip of the moveable needle in the insertion-target region; and control circuitry operably coupled to the at least one sensor and configured to receive information therefrom indicating the skin structure at the insertion-target region in proximity to the distal tip, and the control circuitry configured to output moveable needle targeting instructions to control movement of the moveable needle into the skin in response to one or more signals from the at least one sensor detecting the skin structure in proximity to the distal tip.

In an aspect of the system, the at least one sensor is operable to detect the skin thickness of the vertebrate subject utilizing ultrasound longitudinal waves or ultrasound shear waves. The at least one sensor is operable to detect the skin thickness of the vertebrate subject utilizing optical coherence tomography. The at least one sensor is operable to detect skin conductivity. The skin structure includes skin subsurface structure. The skin subsurface structure includes at least one of size, location, or thickness of dermis, epidermis, hypodermis, papillary region of the dermis, reticular region of the dermis, boundaries between the epidermis and the dermis, boundaries between the dermis and the hypodermis, and boundaries between the reticular and papillary regions.

The needle insertion system includes control circuitry that is operable to control insertion depth of the distal tip of the needle relative to the skin subsurface structure. The control circuitry is operable to control insertion depth of the distal tip of the needle to a specified depth within dermis. The specified depth within the dermis is within a papillary region of the dermis. The specified depth within the dermis is within a reticular region of the dermis. The specified depth within the dermis is proximate to a boundary between a papillary region and a reticular region of the dermis. In an aspect, the control circuitry is operable to control insertion depth of the needle to stop prior to entering hypodermis. In an aspect, the control circuitry is operable to control insertion depth of the needle such that a fluid delivery opening at the distal tip of the needle is at a specified depth within dermis. In an aspect, the control circuitry operably coupled to the at least one sensor is configured to receive location information therefrom about the insertion-target region.

In an aspect of the system, at least one second sensor operable to detect an insertion depth of the distal tip of the movable needle relative to the skin subsurface structure, and to provide one or more signals related to the insertion depth to the control circuitry. The control circuitry is operable to output movable needle targeting instructions to control movement of the moveable needle into the skin in response to the one or more signals related to the insertion depth. The at least one second sensor is operable to detect an insertion depth of the distal tip of the movable needle relative to at least one skin subsurface structure.

A method for inserting a movable needle of a needle insertion system into a vertebrate subject is disclosed that includes locating an insertion-target region on the vertebrate subject with at least one sensor operable to detect skin structure at a position in proximity to the distal tip of the moveable needle in the insertion-target region; outputting location information from the at least one sensor to control circuitry including the location of the insertion-target region and skin structure information in proximity to the distal tip; and automatically moving the needle to a defined depth into skin at the insertion-target region of the vertebrate subject in response to needle-targeting instructions output from the control circuitry. In an aspect, automatically moving the needle to the defined depth into the skin in response to the needle-targeting instructions output from the control circuitry occurs without human intervention and after the needle is in operational range relative to the vertebrate subject. In an aspect, automatically moving the needle to the defined depth into the skin in response to the needle-targeting instructions output from the control circuitry includes automatically moving the needle to the defined depth into the skin with an actuator that receives the needle-targeting instructions. The skin structure includes skin subsurface structure. The skin subsurface structure includes at least one of size, location, or thickness of dermis, epidermis, hypodermis, papillary region of the dermis, reticular region of the dermis, boundaries between the epidermis and the dermis, boundaries between the dermis and the hypodermis, and boundaries between the reticular and papillary regions.

The method includes controlling with the control circuitry insertion depth of the distal tip of the needle relative to the skin subsurface structure. The method includes controlling with the control circuitry insertion depth of the distal tip of the needle to a specified depth within dermis. In an aspect, the specified depth is within a papillary region of the dermis. In an aspect, the specified depth is within a reticular region of the dermis. In an aspect, the specified depth is proximate to a boundary between a papillary region and a reticular region of the dermis. The control circuitry may be configured to control insertion depth of the needle to stop prior to entering hypodermis. The control circuitry may be configured to control insertion depth of the needle such that a fluid delivery opening at the distal tip of the needle is at a specified depth within dermis.

The method includes automatically moving the needle to the defined depth at one or more of an epidermal layer, dermal layer, and hypodermal layer of the skin. In an aspect, the method includes outputting location information from the at least one sensor to the control circuitry including the skin structure information of the size or thickness of dermis, epidermis, hypodermis, papillary region of the dermis, reticular region of the dermis, boundaries between the epidermis and the dermis, boundaries between the dermis and the hypodermis, and boundaries between the reticular region and the papillary region. In an aspect, the method includes locating the insertion-target region on the vertebrate subject with the at least one sensor operable to detect the skin thickness of the vertebrate subject utilizing ultrasound longitudinal waves or ultrasound shear waves. In an aspect, the method includes locating the insertion-target region on the vertebrate subject with the at least one sensor operable to detect the skin thickness of the vertebrate subject utilizing optical coherence tomography. In an aspect, the method includes locating the insertion-target region on the vertebrate subject with the at least one sensor operable to detect skin conductivity.

The method includes outputting location information including an insertion depth of the movable needle from at least one second sensor to the control circuitry. In an aspect, the method includes outputting movable needle targeting instructions from the control circuitry to control movement of the moveable needle into the skin in response to the one or more signals from the least one second sensor related to the insertion depth. In an aspect, the method includes detecting with the least one second sensor an insertion depth of the distal tip of the movable needle relative to at least one skin subsurface structure.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

FIG. 1 depicts a diagrammatic view of an aspect of a needle insertion system.

FIG. 2 depicts a diagrammatic view of an aspect of a needle insertion system.

FIG. 3 depicts a diagrammatic view of an aspect of a needle insertion system.

FIG. 4 depicts a diagrammatic view of an aspect of a needle insertion system.

FIG. 5 depicts a diagrammatic view of an aspect of a needle insertion system.

FIG. 6 depicts a diagrammatic view of an aspect of a needle insertion system.

FIG. 7 depicts a diagrammatic view of an aspect of a needle insertion system.

FIG. 8 depicts a diagrammatic view of an aspect of a needle insertion system.

FIG. 9 depicts a diagrammatic view of an aspect of a needle insertion system.

FIG. 10 depicts a diagrammatic view of a cross section of the skin and underlying structural layers of epithelium and dermis of a vertebrate subject.

FIG. 11 depicts a diagrammatic view of an aspect of a method for inserting a movable needle of a needle insertion system into a vertebrate subject.

FIG. 12 depicts a diagrammatic view of an aspect of a method for inserting a movable needle of a needle insertion system into a vertebrate subject.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented here.

A needle insertion system and a method for inserting a movable hollow needle of a needle insertion system transdermally into a vertebrate subject are disclosed herein. The needle insertion system and the method include one or more sensors for determining skin structure at a position in proximity to the distal tip of the moveable needle at an insertion-target region on the skin of a vertebrate subject and for locating a position for a moveable needle for injection of a medicament. The needle insertion system and the method include control circuitry to move the needle to a defined depth based on the sensed skin structure at the insertion-target region in proximity to the distal tip of the moveable hollow needle prior to injecting the medicament into the vertebrate subject.

A needle insertion system is disclosed that includes a moveable hollow needle having a distal tip configured to be transdermally inserted into a vertebrate subject at an insertion-target region; at least one sensor operable to detect skin structure at a position in proximity to the distal tip of the moveable needle in the insertion-target region; and control circuitry operably coupled to the at least one sensor and configured to receive information therefrom indicating the skin structure at the insertion-target region in proximity to the distal tip, and the control circuitry configured to output moveable needle targeting instructions to control movement of the moveable needle into the skin in response to one or more signals from the at least one sensor detecting the skin structure in proximity to the distal tip.

A method for inserting a movable needle of a needle insertion system into a vertebrate subject is disclosed that includes locating an insertion-target region on the vertebrate subject with at least one sensor operable to detect skin structure at a position in proximity to the distal tip of the moveable needle in the insertion-target region; outputting location information from the at least one sensor to control circuitry including the location of the insertion-target region and skin structure information in proximity to the distal tip; and automatically moving the needle to a defined depth into skin at the insertion-target region of the vertebrate subject in response to needle-targeting instructions output from the control circuitry.

A needle insertion system is disclosed that includes a moveable hollow needle configured to be inserted into a vertebrate subject at an insertion-target region; at least one sensor operable to detect skin structure at a position in proximity to the distal tip of the moveable needle in the insertion-target region; and control circuitry operably coupled to the at least one sensor and configured to receive information therefrom indicating the skin structure at the insertion-target region in proximity to the distal tip, and the control circuitry configured to output moveable needle targeting instructions to control movement of the moveable needle into the skin in response to one or more signals from the at least one sensor detecting the skin structure in proximity to the distal tip. The actuator may include at least one of a pneumatic actuator, a hydraulic actuator, a piezoelectric actuator, a linear actuator, a shape memory actuator, or an electro-mechanical actuator. The actuator may be configured to selectively move the moveable needle in and about x, y, and z axes in response to the sensor determining skin structure and skin substructure at the insertion-target region in proximity to the distal tip. The needle insertion system may include a robotic arm that is moveable in and about x, y, and z axes. The arm is associated with the actuator configured to drive the motion of the arm and configured to selectively move the needle.

A method for inserting a movable needle of a needle insertion system into a vertebrate subject is disclosed that includes locating an insertion-target region on the vertebrate subject with at least one sensor operable to detect skin structure at a position in proximity to the distal tip of the moveable needle in the insertion-target region; outputting location information from the at least one sensor to control circuitry including the location of the insertion-target region and skin structure information in proximity to the distal tip; and automatically moving the needle to a defined depth into skin at the insertion-target region of the vertebrate subject in response to needle-targeting instructions output from the control circuitry. Moving the needle to the defined depth into the skin in response to the needle-targeting instructions output from the control electrical circuitry may occur automatically, e.g., without human intervention, and after the needle is in operational range relative to the vertebrate subject.

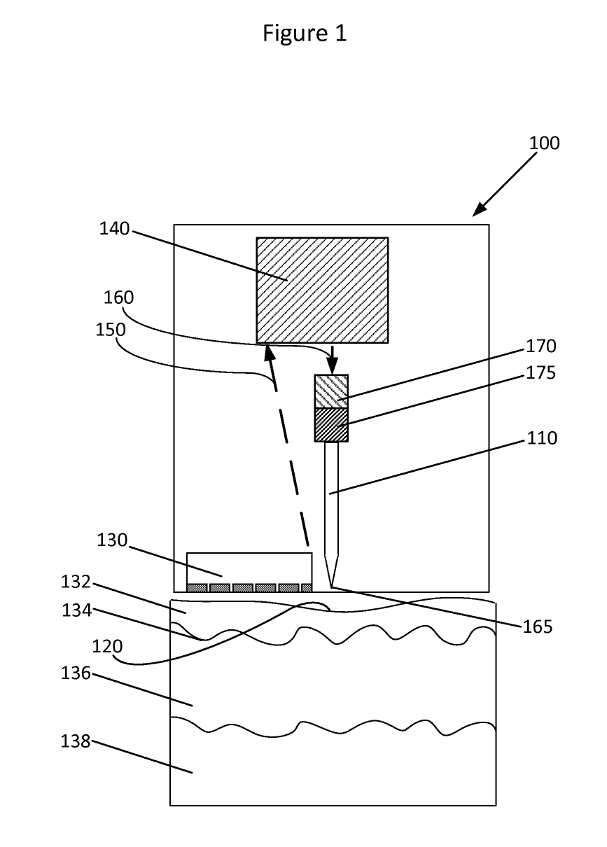

FIGS. 1 through 4 depict a diagrammatic view of an aspect of a needle insertion system. The needle insertion system includes at least one sensor operable to detect skin structure at a position of the moveable needle in the insertion-target region. The control circuitry operable to output moveable needle targeting instructions to control movement of the moveable needle into the skin to a defined target level in the skin structure, for example, at the level of epithelium, papillary dermis, reticular dermis, or hypodermis in response to one or more signals from the at least one sensor indicating the skin structure at the insertion-target region.

FIG. 1 depicts a diagrammatic view of an aspect of a needle insertion system 100 comprising: a moveable hollow needle 110 having a distal tip 165 configured to be transdermally inserted into a vertebrate subject at an insertion-target region 120; at least one sensor 130 operable to detect skin structure 132, 134, 136, 138 at a position in proximity to the distal tip 165 of the moveable needle 110 in the insertion-target region 120; control circuitry 140 operably coupled 150 to the at least one sensor 130 configured to receive information therefrom indicating the skin structure at the insertion-target region 120, and the control circuitry 140 configured to output moveable needle targeting instructions 160 to control movement 170 of the moveable needle 110 into the skin 132, 134, 136, 138 in response to one or more signals 150 from the at least one sensor 130 indicating the skin structure 132, 134, 136, 138 in proximity to the distal tip 165. The needle insertion system 100 includes a medicament reservoir including pump 175 containing medicament to be injected into the specific skin structure 132, 134, 136, 138 by the moveable needle 110 under control of the control circuitry 140.

The control circuitry 140 is operable to control movement 170 of the moveable hollow needle 110 including the distal tip 165 into the skin to an insertion depth 165 in response to one or more signals 150 from the at least one sensor 130 indicating the skin structure 132, 134, 136, 138, and indicating the depth of the skin structure. The skin structure in the insertion-target region includes, for example, dermis 134, 136, epidermis 132, hypodermis 138, papillary region 134 of the dermis, reticular region 136 of the dermis, boundaries 132, 134 between the epidermis and the dermis, boundaries 136, 138 between the dermis and the hypodermis, and boundaries 134, 136 between the reticular and papillary regions. In an embodiment, the moveable needle 110 is not in contact with the skin and is not inserted into the skin or into any layers of the skin structure.

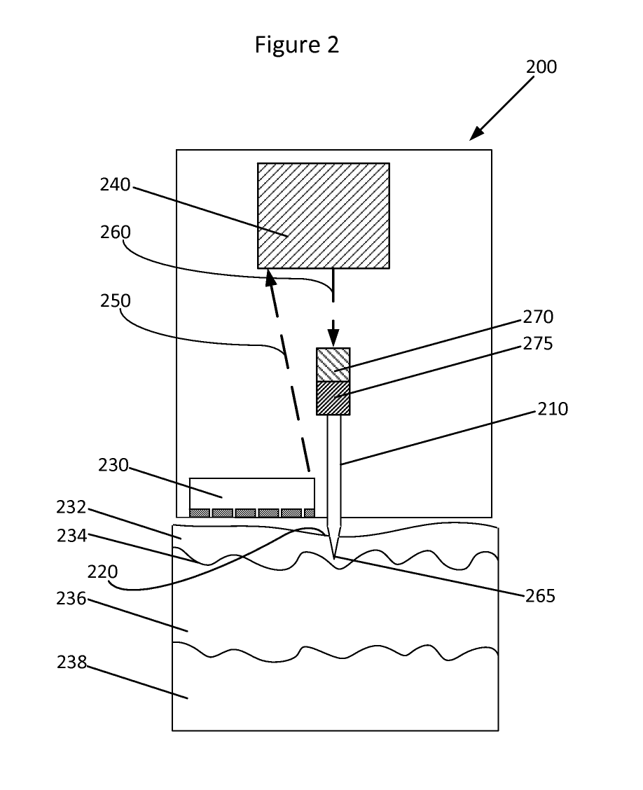

FIG. 2 depicts a diagrammatic view of an aspect of a needle insertion system 200 comprising: a moveable hollow needle 210 having a distal tip 265 configured to be transdermally inserted into a vertebrate subject at an insertion-target region 220; at least one sensor 230 operable to detect skin structure 232, 234, 236, 238 at a position in proximity to the distal tip 265 of the moveable needle 210 in the insertion-target region 220; control circuitry 240 operably coupled 250 to the at least one sensor 230 configured to receive information therefrom indicating the skin structure at the insertion-target region 220, and the control circuitry 240 configured to output moveable needle targeting instructions 260 to control movement 270 of the moveable needle 210 into the skin 232, 234, 236, 238 in response to one or more signals 250 from the at least one sensor 230 indicating the skin structure 232, 234, 236, 238 in proximity to the distal tip 265. The needle insertion system 200 includes a medicament reservoir including pump 275 containing medicament to be injected into the specific skin structure 232, 234, 236, 238 by the moveable needle 210 under control of the control circuitry 240.

The control circuitry 240 is operable to control movement 270 of the moveable hollow needle 210 including the distal tip 265 into the skin to an insertion depth 265 in response to one or more signals 250 from the at least one sensor 230 indicating the skin structure 232, 234, 236, 238 and indicating the depth of the skin structure. In an embodiment, the moveable needle 210 is in contact with the skin and is inserted into the skin at the epidermis layer 232 of the skin structure.

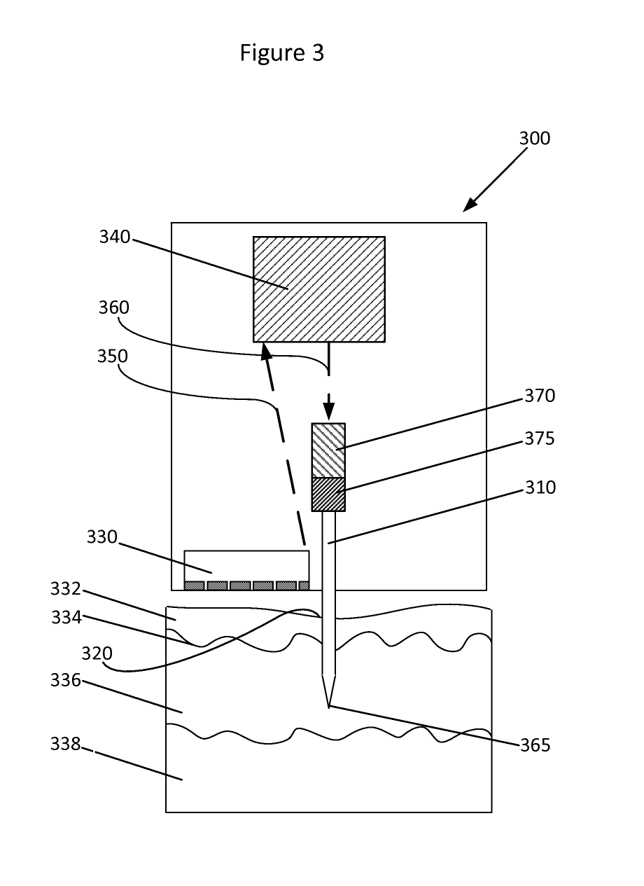

FIG. 3 depicts a diagrammatic view of an aspect of a needle insertion system 300 comprising: a moveable hollow needle 310 having a distal tip 365 configured to be transdermally inserted into a vertebrate subject at an insertion-target region 320; at least one sensor 330 operable to detect skin structure 332, 334, 336, 338 at a position in proximity to the distal tip 365 of the moveable needle 310 in the insertion-target region 320; control circuitry 340 operably coupled 350 to the at least one sensor 330 configured to receive information therefrom indicating the skin structure at the insertion-target region 320, and the control circuitry 340 configured to output moveable needle targeting instructions 360 to control movement 370 of the moveable needle 310 into the skin 332, 334, 336, 338 in response to one or more signals 350 from the at least one sensor 330 indicating the skin structure 332, 334, 336, 338 in proximity to the distal tip 365. The needle insertion system 300 includes a medicament reservoir including pump 375 containing medicament to be injected into the specific skin structure 332, 334, 336, 338 by the moveable needle 310 under control of the control circuitry 340.

The control circuitry 340 is operable to control movement 370 of the moveable hollow needle 310 including the distal tip 365 into the skin to an insertion depth 365 in response to one or more signals 350 from the at least one sensor 330 indicating the skin structure 332, 334, 336, 338 and indicating the depth of the skin structure. In an embodiment, the moveable needle 310 is in contact with the skin and is inserted into the skin at the reticular dermal layer 336 of the skin structure.

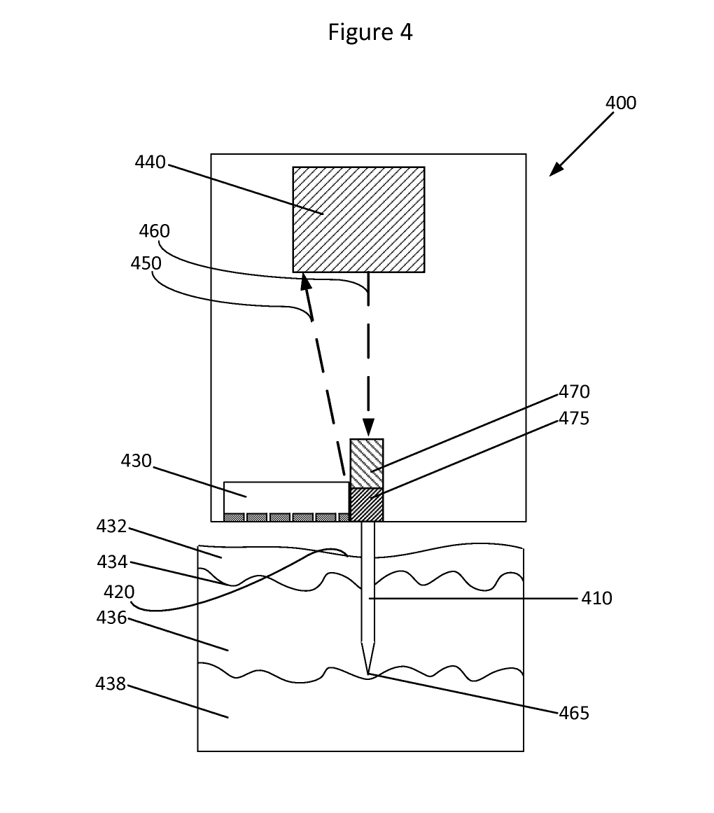

FIG. 4 depicts a diagrammatic view of an aspect of a needle insertion system 400 comprising: a moveable hollow needle 410 having a distal tip 465 configured to be transdermally inserted into a vertebrate subject at an insertion-target region 420; at least one sensor 430 operable to detect skin structure 432, 434, 436, 438 at a position in proximity to the distal tip 465 of the moveable needle 410 in the insertion-target region 420; control circuitry 440 operably coupled 450 to the at least one sensor 430 configured to receive information therefrom indicating the skin structure at the insertion-target region 420, and the control circuitry 440 configured to output moveable needle targeting instructions 460 to control movement 470 of the moveable needle 410 into the skin 432, 434, 436, 438 in response to one or more signals 450 from the at least one sensor 430 indicating the skin structure 432, 434, 436, 438 in proximity to the distal tip 465. The needle insertion system 400 includes a medicament reservoir including pump 475 containing medicament to be injected into the specific skin structure 432, 434, 436, 438 by the moveable needle 410 under control of the control circuitry 440.

The control circuitry 440 is operable to control movement 470 of the moveable hollow needle 410 including the distal tip 465 into the skin to an insertion depth 465 in response to one or more signals 450 from the at least one sensor 430 indicating the skin structure 432, 434, 436, 438 and indicating the depth of the skin structure. In an embodiment, the moveable needle 410 is in contact with the skin and is inserted into the skin at the border of the reticular dermal layer 436 and the hypodermal layer 438 of the skin structure.

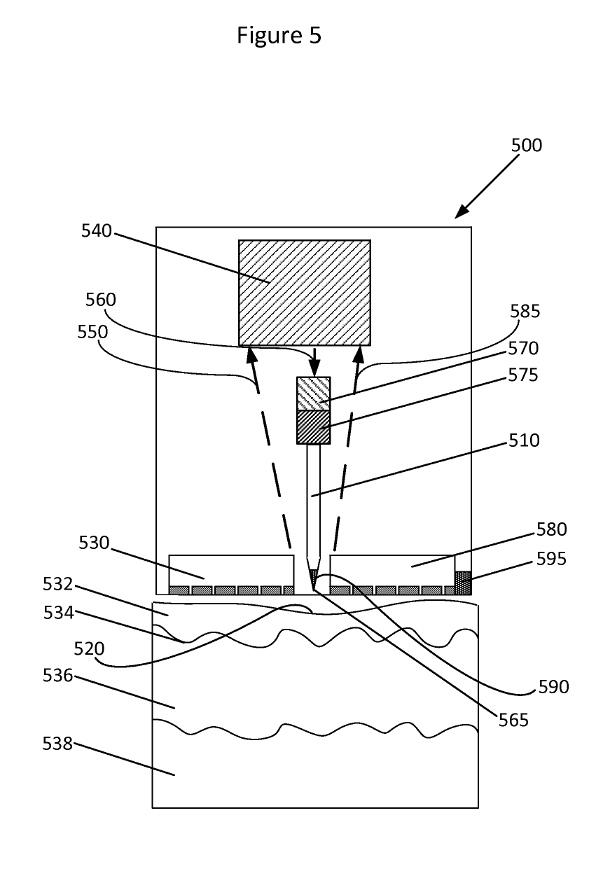

FIGS. 5 through 8 depict a diagrammatic view of an aspect of a needle insertion system. The needle insertion system includes at least one sensor and at least one second sensor. The at least one sensor is operable to detect skin structure at a position of the moveable needle in the insertion-target region utilizing one or more of ultrasound (ultrasound longitudinal waves or ultrasound shear waves), optical coherence tomography, or skin conductivity. The control circuitry operable to output moveable needle targeting instructions to control movement of the moveable needle into the skin to a defined target level in the skin structure, for example, at the level of epithelium, papillary dermis, reticular dermis, or hypodermis in response to one or more signals from the at least one sensor indicating the skin structure at the insertion-target region. The control circuitry is further operably coupled to at least one second sensor. The at least one second sensor is operable to detect an insertion depth of the movable needle, and to provide one or more signals related to the insertion depth to the control circuitry, for example, the one or more signals may represent a measurement of impedance.

In an embodiment, the at least one second sensor is operable to detect an insertion depth of the movable needle utilizing ultrasound, e.g., ultrasound longitudinal waves or ultrasound shear waves, and utilizing an ultrasound detector to determine the insertion depth of the movable needle. In an embodiment, the at least one second sensor is operable to detect an insertion depth of the movable needle by utilizing a mechanical, electrical (capacitance measurement), or optical encoder to determine how far the needle has moved relative to support structures and mechanical structures for the moveable needle. See, e.g., U.S. Pat. No. 6,951,549, which is incorporated herein by reference. The movement of the needle to an insertion depth can then be referenced to a skin-structure location by having the at least one sensor operable to detect skin structure referenced to a position of the support structures and mechanical structures of the needle.

FIG. 5 depicts a diagrammatic view of an aspect of a needle insertion system 500 comprising: a moveable hollow needle 510 having a distal tip 565 configured to be transdermally inserted into a vertebrate subject at an insertion-target region 520; at least one sensor 530 operable to detect skin structure 532, 534, 536, 538 at a position in proximity to the distal tip 565 of the moveable needle 510 in the insertion-target region 520; control circuitry 540 operably coupled 550 to the at least one sensor 530 configured to receive information therefrom indicating the skin structure at the insertion-target region 520, and the control circuitry 540 configured to output moveable needle targeting instructions 560 to control movement 570 of the moveable needle 510 into the skin 532, 534, 536, 538 in response to one or more signals 550 from the at least one sensor 530 indicating the skin structure 532, 534, 536, 538 in proximity to the distal tip 565. The needle insertion system 500 includes a medicament reservoir including a pump 575 containing medicament to be injected into the specific skin structure 532, 534, 536, 538 by the moveable needle 510 under control of the control circuitry 540.

The control circuitry 540 is further operably coupled to at least one second sensor 580. The at least one second sensor 580 is operable to detect an insertion depth of the distal tip 565 of the movable needle 510. The at least one second sensor 580 is operable to provide one or more signals 585 related to the insertion depth of the distal tip 565 to the control circuitry 540, for example, the one or more signals may represent a measurement of impedance. The at least one second sensor 580 may include a first electrode 590, e.g., located at a distal end of the needle, and a second electrode 595, e.g., located at a distal end of the needle insertion system. The at least one second sensor 580 may output an electronic signal 585 associated with an impedance measurement between the first electrode 590 and the second electrode 595. The control circuitry 540 is operable to output movable needle targeting instructions 560 to control movement of the moveable needle 510 into the skin in response to the one or more impedance measurement signals 585 that correlate to the insertion depth of the distal tip 565 of the moveable needle based on the impedance measurement signals 585 between the first electrode 590 and the second electrode 595. The at least one second sensor 580 is operable to detect an insertion depth of the distal tip 565 of the movable needle 510 relative to at least one skin subsurface structure 532, 534, 536, 538. The control circuitry 540 in response to signals from the at least one sensor 530 and the at least one second sensor 580 can target the needle to a prescribed depth of the distal tip 565 into the target tissue at the insertion/target region 520. In an embodiment, based on the impedance measurement signals 585, the insertion depth of the distal tip 565 of the movable needle 510 is zero relative to the at least one skin subsurface structure 532, 534, 536, 538. The control circuitry 540 may also provide electronic and/or automatic feedback associated with the validity and/or administration of an injection event based on the impedance measurement signals 585 between the first electrode 590 and the second electrode 595.

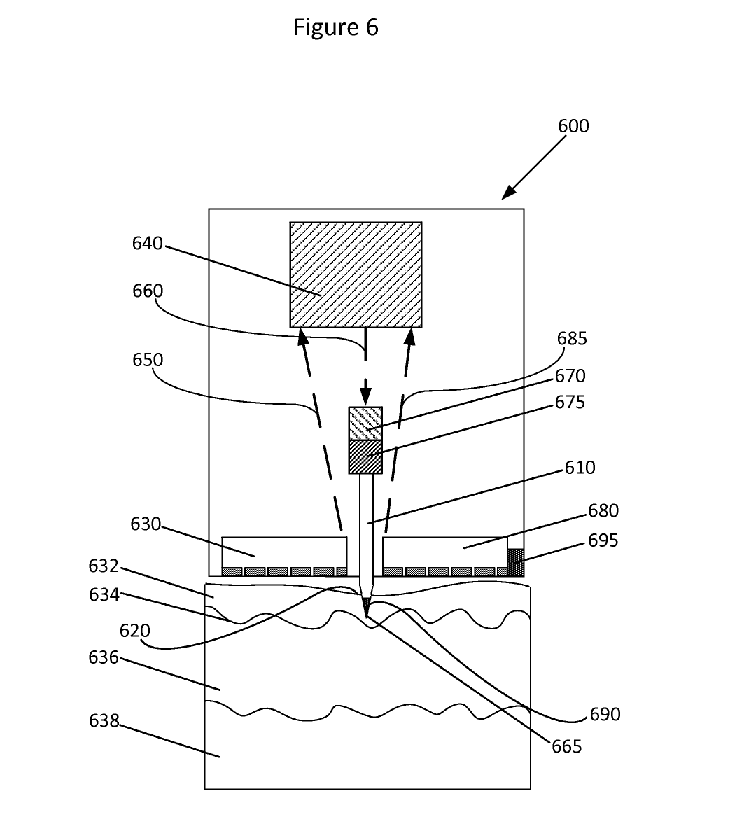

FIG. 6 depicts a diagrammatic view of an aspect of a needle insertion system 600 comprising: a moveable hollow needle 610 having a distal tip 665 configured to be transdermally inserted into a vertebrate subject at an insertion-target region 620; at least one sensor 630 operable to detect skin structure 636, 634, 636, 638 at a position in proximity to the distal tip 665 of the moveable needle 610 in the insertion-target region 620; control circuitry 640 operably coupled 650 to the at least one sensor 630 configured to receive information therefrom indicating the skin structure at the insertion-target region 620, and the control circuitry 640 configured to output moveable needle targeting instructions 660 to control movement 670 of the moveable needle 610 into the skin 632, 634, 636, 638 in response to one or more signals 650 from the at least one sensor 630 indicating the skin structure 632, 634, 636, 638 in proximity to the distal tip 665. The needle insertion system 600 includes a medicament reservoir including pump 675 containing medicament to be injected into the specific skin structure 632, 634, 636, 638 by the moveable needle 610 under control of the control circuitry 640.

The control circuitry 640 is further operably coupled to at least one second sensor 680. The at least one second sensor 680 is operable to determine a location within the insertion-target region and to detect an insertion depth of the distal tip 665 of the movable needle 610. The at least one second sensor 680 is operable to provide one or more signals 685 related to the insertion depth of the distal tip 665 to the control circuitry 640, for example, the one or more signals may represent a measurement of impedance. The at least one second sensor 680 may include a first electrode 690, e.g., located at a distal end of the needle, and a second electrode 695, e.g., located at a distal end of the needle insertion system. The at least one second sensor 680 may output an electronic signal 685 associated with an impedance measurement between the first electrode 690 and the second electrode 695. The control circuitry 640 is operable to output movable needle targeting instructions 660 to control movement of the moveable needle 610 into the skin in response to the one or more impedance measurement signals 685 that correlate to the insertion depth of the distal tip 665 of the moveable needle based on the impedance measurement signals 685 between the first electrode 690 and the second electrode 695. The at least one second sensor 680 is operable to detect an insertion depth 665 of the movable needle 610 relative to at least one skin subsurface structure 632, 634, 636, 638. The control circuitry 640 in response to signals from the at least one sensor 630 and the at least one second sensor 680 can target the needle to a prescribed depth of the distal tip 665 into the target tissue at the insertion/target region 620. In an embodiment, based on the impedance measurement signals 685, the insertion depth of the distal tip 665 of the movable needle 610 penetrates to the epithelium 632 into the target tissue at the insertion/target region 620. The control circuitry 640 may also provide electronic and/or automatic feedback associated with the validity and/or administration of an injection event based on the impedance measurement signals 685 between the first electrode 690 and the second electrode 695.

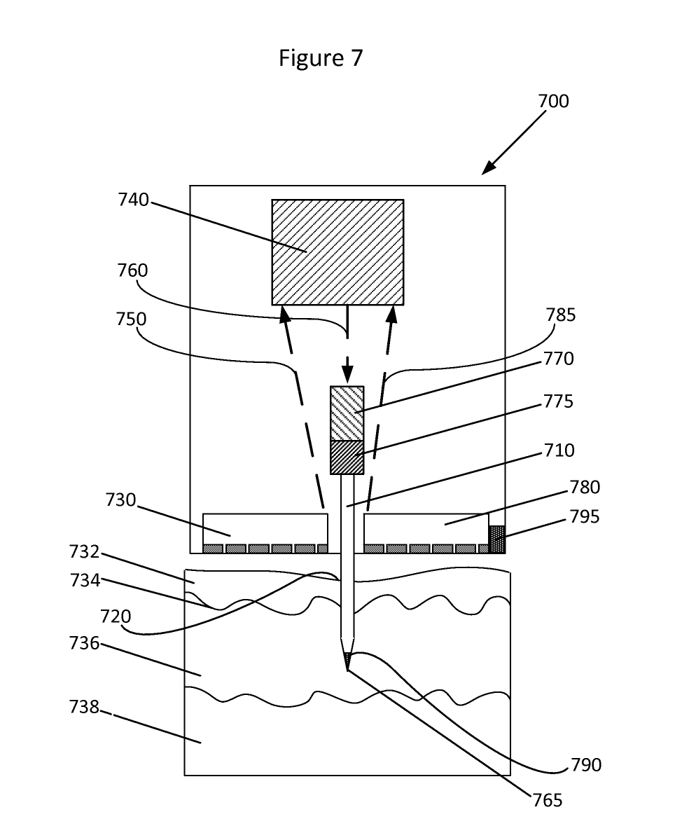

FIG. 7 depicts a diagrammatic view of an aspect of a needle insertion system 700 comprising: a moveable hollow needle 710 having a distal tip 765 configured to be transdermally inserted into a vertebrate subject at an insertion-target region 720; at least one sensor 730 operable to detect skin structure 733, 734, 736, 738 at a position in proximity to the distal tip 765 of the moveable needle 710 in the insertion-target region 720; control circuitry 740 operably coupled 750 to the at least one sensor 730 configured to receive information therefrom indicating the skin structure at the insertion-target region 720, and the control circuitry 740 configured to output moveable needle targeting instructions 760 to control movement 770 of the moveable needle 710 into the skin 732, 734, 736, 738 in response to one or more signals 750 from the at least one sensor 730 indicating the skin structure 732, 734, 736, 738 in proximity to the distal tip 765. The needle insertion system 700 includes a medicament reservoir including pump 775 containing medicament to be injected into the specific skin structure 732, 734, 736, 738 by the moveable needle 710 under control of the control circuitry 740.

The control circuitry 740 is further operably coupled to at least one second sensor 780. The at least one second sensor 780 is operable to determine a location of the movable needle 710 within the insertion-target region and to detect an insertion depth of distal tip 765 of the movable needle 710. The at least one second sensor 780 is operable to provide one or more signals 785 related to the insertion depth of the distal tip 765 to the control circuitry 740, for example, the one or more signals may represent a measurement of impedance. The at least one second sensor 780 may include a first electrode 790, e.g., located at a distal end of the needle, and a second electrode 795, e.g., located at a distal end of the needle insertion system. The at least one second sensor 780 may output an electronic signal 785 associated with an impedance measurement between the first electrode 790 and the second electrode 795. The control circuitry 740 is operable to output movable needle targeting instructions 760 to control movement of the moveable needle 710 into the skin in response to the one or more impedance measurement signals 785 that correlate to the insertion depth of the distal tip 765 of the moveable needle based on the impedance measurement signals 785 between the first electrode 790 and the second electrode 795. The at least one second sensor 780 is operable to detect an insertion depth of the distal tip 765 of the movable needle 710 relative to at least one skin subsurface structure 732, 734, 736, 738. The control circuitry 740 in response to signals from the at least one sensor 730 and the at least one second sensor 780 can target the needle to a prescribed depth of the distal tip 765 into the target tissue at the insertion/target region 720. In an embodiment, based on the impedance measurement signals 785, the insertion depth of the distal tip 765 of the movable needle 710 penetrates to the reticular dermis 736 into the target tissue at the insertion/target region 720. The control circuitry 740 may also provide electronic and/or automatic feedback associated with the validity and/or administration of an injection event based on the impedance measurement signals 785 between the first electrode 790 and the second electrode 795.

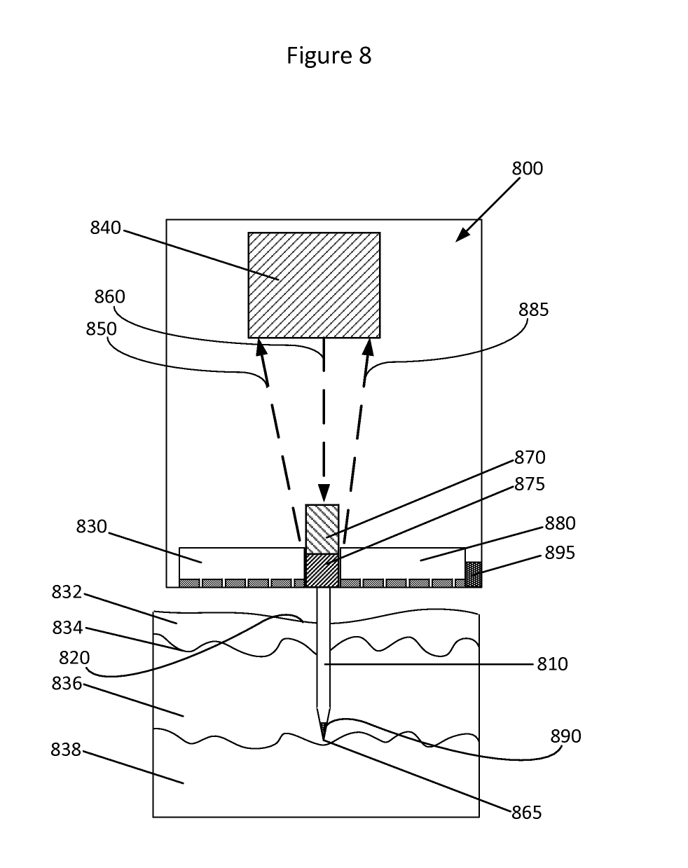

FIG. 8 depicts a diagrammatic view of an aspect of a needle insertion system 800 comprising: a moveable hollow needle 810 having a distal tip 865 configured to be transdermally inserted into a vertebrate subject at an insertion-target region 820; at least one sensor 830 operable to detect skin structure 832, 834, 836, 838 at a position in proximity to the distal tip 865 of the moveable needle 810 in the insertion-target region 820; control circuitry 840 operably coupled 850 to the at least one sensor 830 configured to receive information therefrom indicating the skin structure at the insertion-target region 820, and the control circuitry 840 configured to output moveable needle targeting instructions 860 to control movement 870 of the moveable needle 810 into the skin 832, 834, 836, 838 in response to one or more signals 850 from the at least one sensor 830 indicating the skin structure 832, 834, 836, 838 in proximity to the distal tip 865. The needle insertion system 800 includes a medicament reservoir including pump 875 containing medicament to be injected into the specific skin structure 832, 834, 836, 838 by the moveable needle 810 under control of the control circuitry 840.

The control circuitry 840 is further operably coupled to at least one second sensor 880. The at least one second sensor 880 is operable to determine a location of the movable needle 810 within the insertion-target region and to detect an insertion depth of the distal tip 865 of the movable needle 810. The at least one second sensor 880 is operable to provide one or more signals 885 related to the insertion depth of the distal tip 865 to the control circuitry 840, for example, the one or more signals may represent a measurement of impedance. The at least one second sensor 880 may include a first electrode 890, e.g., located at a distal end of the needle, and a second electrode 895, e.g., located at a distal end of the needle insertion system. The at least one second sensor 880 may output an electronic signal 885 associated with an impedance measurement between the first electrode 890 and the second electrode 895. The control circuitry 840 is operable to output movable needle targeting instructions 860 to control movement of the moveable needle 810 into the skin in response to the one or more impedance measurement signals 885 that correlate to the insertion depth 865 of the moveable needle based on the impedance measurement signals 885 between the first electrode 890 and the second electrode 895. The at least one second sensor 880 is operable to detect an insertion depth of the distal tip 865 of the movable needle 810 relative to at least one skin subsurface structure 832, 834, 836, 838. The control circuitry 840 in response to signals from the at least one sensor 830 and the at least one second sensor 880 can target the needle to a prescribed depth of the distal tip 865 into the target tissue at the insertion/target region 820. In an embodiment, based on the impedance measurement signals 885, the insertion depth of the distal tip 865 of the movable needle 810 penetrates to the border of the reticular dermis and hypodermis 836, 838 into the target tissue at the insertion/target region 820. The control circuitry 840 may also provide electronic and/or automatic feedback associated with the validity and/or administration of an injection event based on the impedance measurement signals 885 between the first electrode 890 and the second electrode 895.

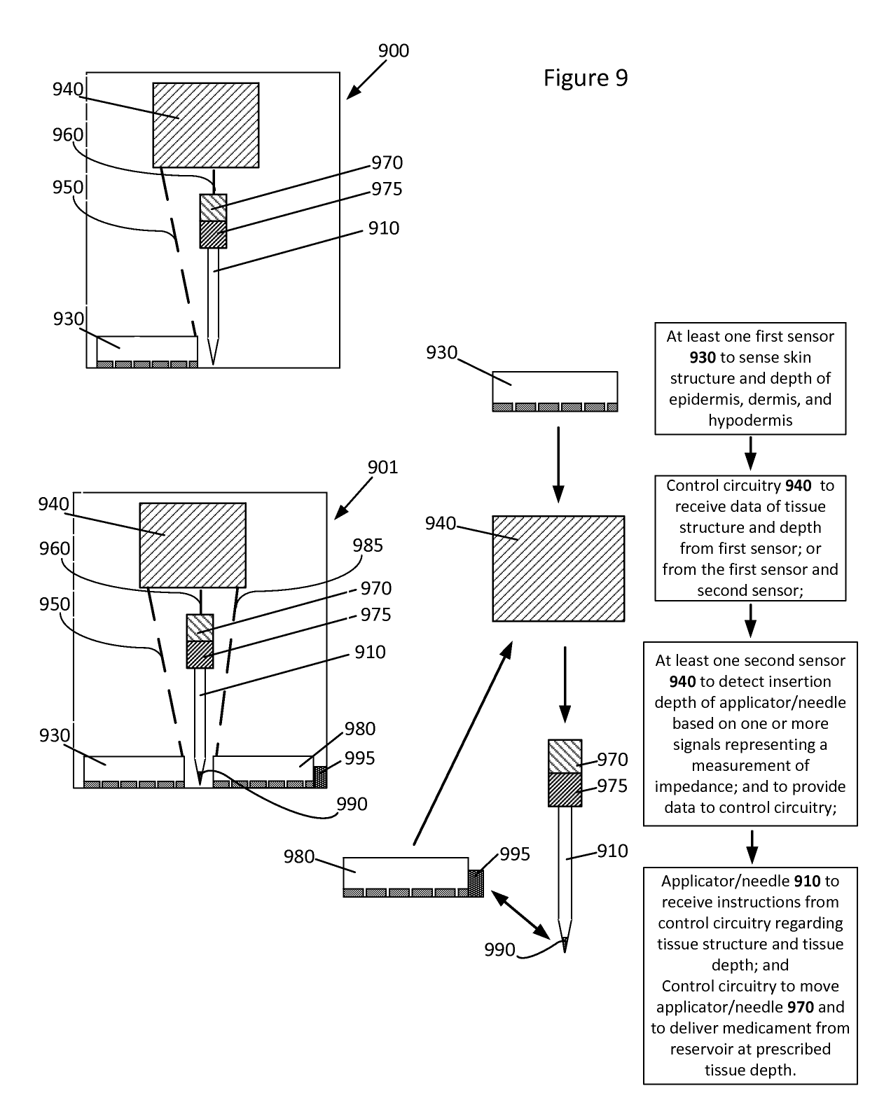

FIG. 9 depicts a diagrammatic view of an aspect of a needle insertion system 900 and 910. A needle insertion system 900 includes at least one sensor 930. At least one sensor 930 is operable to detect skin structure at a position of the moveable hollow needle 910 in the insertion-target region; control circuitry 940 operably coupled 950 to the at least one sensor 903 configured to receive information therefrom indicating the skin structure at the insertion-target region, and the control circuitry 940 configured to output moveable needle targeting instructions 960 to control movement 970 of the moveable needle 910 attached to reservoir 975 into the skin in response to one or more signals 950 from the at least one sensor 930 indicating the skin structure.

A needle insertion system 901 includes at least one first sensor 930 and at least one second sensor 980. The at least one sensor 930 is operable to detect skin structure at a position of the moveable hollow needle 910 in the insertion-target region; control circuitry 940 operably coupled 950 to the at least one sensor 903 configured to receive information therefrom indicating the skin structure at the insertion-target region, and the control circuitry 940 configured to output moveable needle targeting instructions 960 to control movement 970 of the moveable needle 910 into the skin in response to one or more signals 950 from the at least one sensor 930 indicating the skin structure. The at least one second sensor 980 is operable to detect an insertion depth of the movable needle 910, and to provide one or more signals 985 related to the insertion depth to the control circuitry 940, for example, the one or more signals may represent a measurement of impedance. The at least one second sensor 980 may include a first electrode 990, e.g., located at a distal end of the needle, and a second electrode 995, e.g., located at a distal end of the needle insertion system. The at least one second sensor 980 may output an electronic signal 985 associated with an impedance measurement between the first electrode 990 and the second electrode 995.

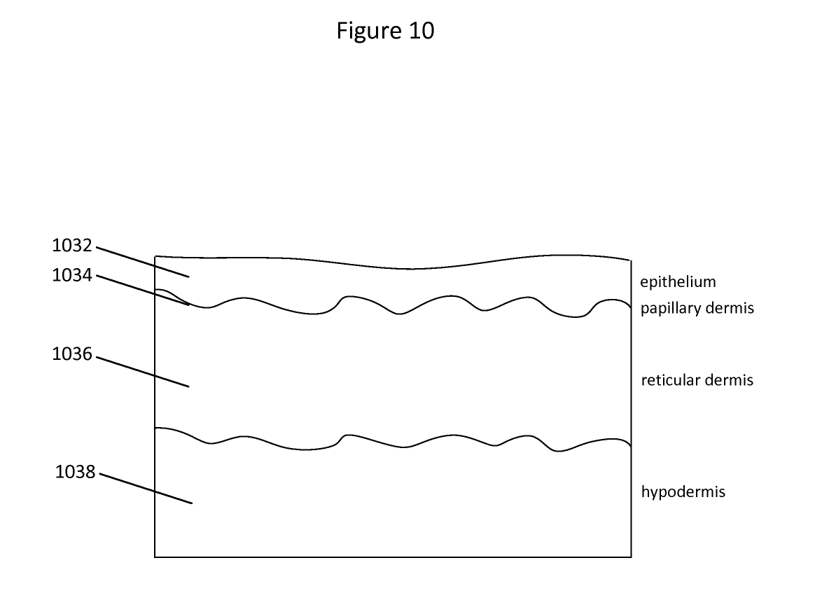

FIG. 10 shows a diagrammatic view of a cross section of the skin and underlying structural layers of epithelium and dermis of a vertebrate subject. The skin subsurface structure includes at least one of size, location, or thickness of dermis 1034, 1036, epidermis 1032, hypodermis 1038, papillary region 1034 of the dermis, reticular region 1036 of the dermis, boundaries 1032, 1034 between the epidermis and the dermis, boundaries 1036, 1038 between the dermis and the hypodermis, and boundaries 1034, 1036 between the reticular and papillary regions. The dermis 1034, 1036 is a layer of skin between the epidermis/epithelium 1032 (with which it makes up the cutis) and subcutaneous tissues, that consist of connective tissue and cushion the body from stress and strain. The dermis is divided into two layers, the superficial area adjacent to the epidermis called the papillary region 1034 and a deep thicker area known as the reticular dermis 1036. The dermis 1034, 1036 is tightly connected to the epidermis 1032 through a basement membrane. Structural components of the dermis are collagen, elastic fibers, and extrafibrillar matrix. The dermis 1034, 1036 also contains mechanoreceptors that provide the sense of touch and heat, hair follicles, sweat glands, sebaceous glands, apocrine glands, lymphatic vessels and blood vessels. Those blood vessels provide nourishment and waste removal for both dermal 1034, 1036 and epidermal cells 1032.

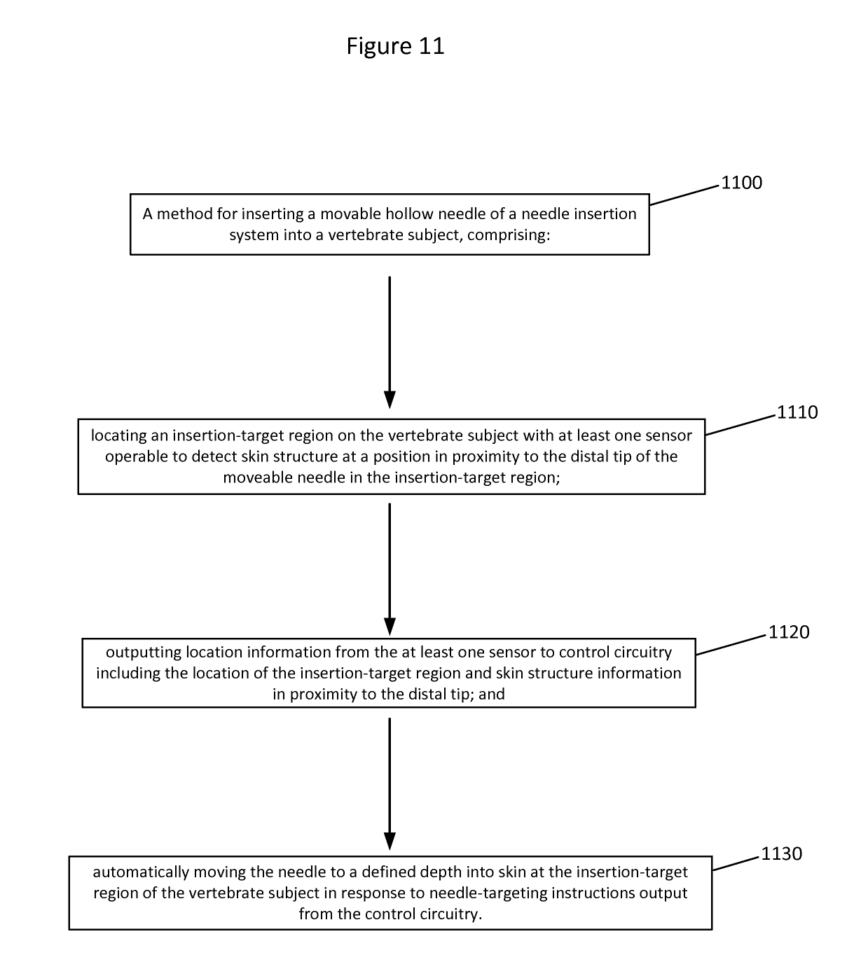

FIG. 11 depicts a diagrammatic view of an aspect of a method 1100 for inserting a movable needle of a needle insertion system into a vertebrate subject. A method 1100 for inserting a movable needle of a needle insertion system into a vertebrate subject is disclosed that includes locating 1110 an insertion-target region on the vertebrate subject with at least one sensor operable to detect skin structure at a position in the insertion-target region; outputting 1120 location information from the at least one sensor to control circuitry including the location of the insertion-target region and skin structure information; and automatically moving 1130 the needle to a defined depth into skin at the insertion-target region of the vertebrate subject in response to needle-targeting instructions output from the control circuitry.

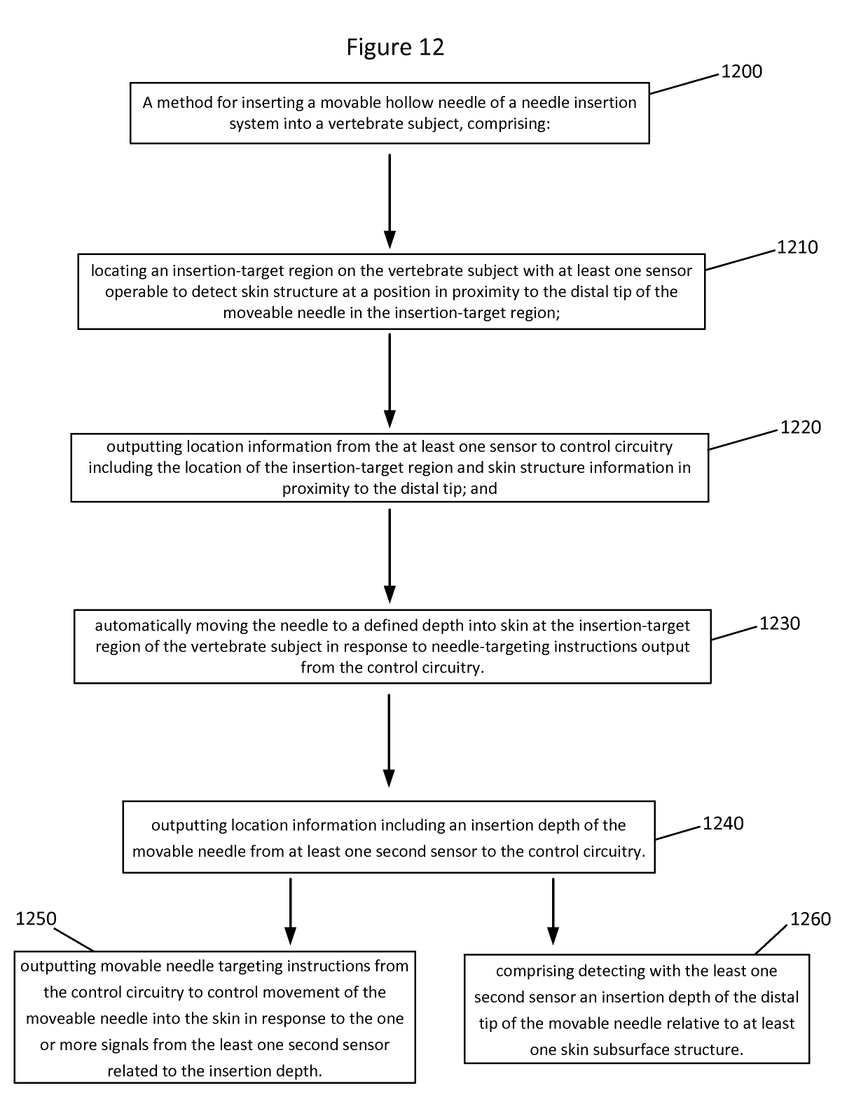

FIG. 12 depicts a diagrammatic view of an aspect of a method 1200 for inserting a movable needle of a needle insertion system into a vertebrate subject. A method 1200 for inserting a movable needle of a needle insertion system into a vertebrate subject is disclosed that includes locating 1210 an insertion-target region on the vertebrate subject with at least one sensor operable to detect skin structure at a position in the insertion-target region; outputting 1220 location information from the at least one sensor to control circuitry including the location of the insertion-target region and skin structure information; and automatically moving 1230 the needle to a defined depth into skin at the insertion-target region of the vertebrate subject in response to needle-targeting instructions output from the control circuitry. The method 1200 for inserting a movable needle of a needle insertion system further includes outputting 1240 location information including an insertion depth of the movable needle from at least one second sensor to control circuitry. The method 1200 further includes outputting 1250 movable needle targeting instructions from the control circuitry to control movement of the moveable needle into the skin in response to the one or more signals from the least one second sensor related to the insertion depth. The method 1200 further includes detecting 1260 with the least one second sensor an insertion depth of the movable needle relative to at least one skin subsurface structure.

### Sensors for Ultrasonic Scanning of Skin Structure

The needle insertion system includes one or more sensors operable to detect skin structure at a position of the moveable needle in the insertion-target region. The one or more sensors are operable to detect the skin thickness of the vertebrate subject utilizing ultrasound longitudinal waves or ultrasound shear waves.

Ultrasound scanning is an important diagnostic tool in dermatology. There are 2 basic types of ultrasonography with dermatologic applications. The best established is 20-MHz scanning, which can be used to measure skin thickness of the epidermal and dermal layers. Real-time sonography with 7.5- to 10-MHz probes has assumed an increasingly important role, since it is used to search for and image cutaneous and subcutaneous tissues in a variety of clinical settings. Ultrasonography is capable of revealing the 3-dimensional size and outline of subcutaneous lesions, for example, lymph nodes, subcutaneous tissues, and their relation to adjacent vessels. In addition to conventional B-mode sonography, additional ultrasound techniques such as native and signal-enhanced color Doppler sonography can be used to assess subcutaneous tissues such as blood vessels and peripheral lymph nodes.

Ultrasound scanning is of importance in many aspects of clinical medicine. As a noninvasive diagnostic method, 5- to 10-MHz real-time B-mode sonography has been successfully applied. Diagnostic ultrasound has also entered the arena of clinical dermatology. High-frequency ultrasound systems using at least 20-MHz probes has been used for clinical dermatology. These systems provide information about the axial and lateral extension of the epidermal, dermal, and subdermal structure in addition to tumoral and inflammatory processes of the skin and the subcutaneous fatty tissue and, therefore, are of special interest in preoperative situations and for the monitoring of skin conditions under therapy.

In contrast to the role of the high-frequency ultrasound systems, the use of ultrasound scanning using 7.5- to 10-MHz probes has provided results from specialized diagnostic units. Technical aspects, examination techniques, and different ultrasound methods such as B-mode sonography, native color Doppler sonography (CDS), and signal enhanced CDS, may be used for analysis of epidermal, dermal, and subdermal structure.

Ultrasound is defined as energy above 20 kHz, which represents the upper frequency limit of human hearing. Transducers, which are thin disk-shaped crystals made out of piezoelectric materials, generate acoustic energy when a voltage is applied to them. Kilohertz to megahertz acoustic vibrations (frequencies) are generated when those piezoelectric materials expand and contract. Transducers may be produced from a variety of materials including, but not limited to quartz, lithium sulfate, ceramics, and plastic polymers. These substances have allowed the development of transducers that produce higher frequencies, which are of special interest for dermatologists because the wavelengths of higher frequencies are smaller and, therefore, allow better resolution of small objects located near the skin surface. With increasing frequency, the depth of penetration of ultrasound waves decreases; for example, ultrasound units using 20 MHz only penetrate 8 mm. Transducers used earlier in general medicine for diagnostic purposes used frequencies between 2 and 5 MHz. At present, the transducers for the diagnosis of regional lymph nodes and soft tissue tumors operate in the 7.5- to 10-MHz range, while the high-frequency transducers function in the 20- to 50-MHz range and may be used to evaluate cutaneous structures of skin subsurface structure including epidermal and dermal structures.

Diagnostic ultrasound systems may be based on pulse echo systems, similar to radar or sonar technology. Acoustic energy is emitted from the transducer. The expansion and contraction of the transducer is transferred as a pulse to the adjacent fluid or tissue and propagates as a wave, which can be reflected or refracted at tissue boundaries. The echo (returning wave) reaches the transducer during breaks of impulse generation. The vibration of the transducer caused by the returning wave generates a voltage difference over the electrodes. These echoes are converted by the transducer into signals that are processed and stored by the computer system.

Resolution of ultrasound systems may refer to either axial or lateral resolution. The axial resolution is the smallest thickness that can be measured and is related to the duration of a pulse. The lateral resolution refers to the width of the smallest structures that can be resolved and is related to the width of the beam at the focus zone. In general, ultrasound systems convert the voltage changes recorded by the transducer and display these signals as images. Two different types of signal processing can be distinguished: A-scans and B-scans. A-scans depict the magnitude of reflection along a single line, resulting in a graph that shows changes in amplitude relative to time. The time of transit of the acoustic wave correlates with distance. Echoes occur at boundaries between tissues where there is a change of acoustic impedance. A-scan ultrasound systems may be mainly used in ophthalmology. B-scans combine the information from sequential single A-scans and display each point according to its relative brightness (hence B-scan). Each point on a B-scan is brighter or darker, corresponding to the intensity of echoes from the corresponding anatomic structure. Therefore, B-scans provide images that resemble anatomic cross sections of scanned tissues. B-mode ultrasonography may include gray-scale images which displays the amplitudes of signals received from reflected ultrasound signals. Currently, B-mode scans are a mainstay of all ultrasonographic procedures in dermatology using intermediate- or high-frequency ultrasound systems. See, e.g., Schmid-Wendtner, et al., Arch Dermatol., 141: 217-224, 2005. Downloaded from: http://archderm.jamanetwork.com/on 03/13/2014, which is incorporated herein by reference.

Supersonic shear imaging (SSI) is an ultrasound-based technique for real-time visualization of soft tissue viscoelastic properties, for example, skin subsurface structure including epidermal and dermal structures. Using ultrasonic focused beams, it is possible to remotely generate mechanical vibration sources radiating low-frequency, shear waves inside epidermal and dermal tissues. Relying on this concept, SSI proposes to create such a source and make it move at a supersonic speed. In analogy with the “sonic boom” created by a supersonic aircraft, the resulting shear waves will interfere constructively along a Mach cone, creating two intense plane shear waves. These waves propagate through the medium and are progressively distorted by tissue heterogeneities. An ultrafast scanner prototype is able to both generate this supersonic source and image (5000 frames/s) the propagation of the resulting shear waves. Using inversion algorithms, the shear elasticity of medium can be mapped quantitatively from this propagation movie. The SSI enables tissue elasticity mapping in less than 20 ms, even in strongly viscous medium. Modalities such as shear compounding are implementable by tilting shear waves in different directions and improving the elasticity estimation. Results validating SSI in heterogeneous phantoms are presented. The clinical applicability of SSI applies to detection of skin subsurface structure including epidermal and dermal structures. See, e.g., Bercoff et al., IEEE Transactions on Ultrasonics, Ferroelectrics, and Frequency Control 51: 396-409, 2004, which is incorporated herein by reference.

A technique for inducing simultaneous ultrafast imaging shear waves in soft tissues, including cutaneous structures of skin subsurface structure including epidermal, dermal, and subdermal structures, has been studied and applied in vitro and in vivo. Quantitative elasticity maps have been achieved, particularly in heterogeneous medium. Based on the acoustic radiation force, supersonic shear imaging (SSI) relies on the generation of a supersonic moving source radiating shear waves in the body. Such a supersonic regime enables the computation of a quantitative elasticity map of an organ in a few milliseconds, even in strongly viscous media. Insensitive to patient motion or boundary conditions, this technique requires only a classical ultrasonic transducer array and should be able to provide elasticity maps of the scanned regions without any technical addition. Studies may focus on providing viscosity maps of the medium, giving the physician a complete cartography of the viscoelastic properties of human tissues. The validation of SSI will be useful for determination of epidermal, dermal, and subdermal structure of a vertebrate subject. See, e.g., Bercoff et al., IEEE Transactions on Ultrasonics, Ferroelectrics, and Frequency Control 51: 396-409, 2004, which is incorporated herein by reference.

### Sensors for Optical Coherence Tomography (OCT) Scanning of Skin Structure

A needle insertion system includes at least one sensor operable to detect skin structure at a position of the moveable needle in the insertion-target region. Optical Coherence Tomography (OCT) is a noninvasive optical imaging modality that provides real-time, 1D depth, 2D cross-sectional, and 3D volumetric images with micron-level resolution and millimeters of imaging depth of skin subsurface structure including epidermal, dermal, and subdermal structures. OCT images provide structural information of a sample, based on light backscattered from different layers of skin subsurface structure within the tissue. Although it is considered to be the optical analog to ultrasound, OCT achieves higher resolution through the use of near infrared wavelengths, at the cost of decreased penetration depth. In addition to high resolution, the non-contact, noninvasive advantage of OCT makes it well suited for imaging samples such as biological tissue including epidermal, dermal, and subdermal structure of a subject.

Swept Source Optical Coherence Tomography (SS-OCT) System is based on a Micro-Electro-Mechanical (MEMS)-tunable Vertical Cavity Surface Emitting Laser (VCSEL) that is specifically designed for optimal performance in OCT applications. (OCS1310V1 Swept Source Optical Coherence Tomography (SS-OCT) System. Thorlabs, Inc.) The MEMS-VCSEL OCT system provides high-speed imaging at imaging depth range of 12 mm. The 1300 nm central wavelength and greater than 100 mm coherence length of this swept laser source enable imaging through highly scattering samples with an imaging range of 12 mm (current imaging range capability is solely limited by data acquisition electronics). The system includes a 100 kHz MEMS-VCSEL Benchtop Laser Source, handheld probe, probe stand, and computer with user software. See, e.g., ThorLabs, Inc. http://www.thorlabs.com/newgrouppage9.cfm?objectgroup_id=6473&pn=OCS1310V1, which is incorporated herein by reference.

Using optical coherence tomography (OCT) for deep imaging in skin, significant optical clearing may be achieved by topical application of an optical clearing agent PEG-400, a chemical enhancer (thiazone or propanediol), and physical massage for approximately 15 minutes. When all three components were applied together a 15 min treatment may achieve a three-fold increase in the optical coherence tomography OCT reflectance from a 300 μm depth and 31% enhancement in image depth Z_{threshold}. The strong optical scattering of skin tissue makes it difficult for optical coherence tomography to achieve deep imaging in skin in the absence of pre-treatment of the skin. See e.g., Wen et al., J. Biomedical Optics 17(6), 066022 (June 2012) available online at: www.SPIEDigitalLibrary.org/jbo, which is incorporated herein by reference.

The needle insertion system includes at least one sensor operable to detect skin structure at a position of the moveable needle in the insertion-target region. The sensor may determine the skin structure utilizing a variety of methods including, but not limited to, ultrasound longitudinal waves, ultrasound shear waves, or optical coherence tomography. The detected skin structure includes skin subsurface structure, including at least one of size, location, or thickness of dermis, epidermis, hypodermis, papillary region of the dermis, reticular region of the dermis, boundaries between the epidermis and the dermis, boundaries between the dermis and the hypodermis, and boundaries between the reticular and papillary regions. See e.g., U.S. Pat. No. 6,135,994 issued to Chernoff on Oct. 24, 2000 and U.S. Pat. No. 7,722,535 issued to Randlov et al. on May 25, 2010, Schmid-Wendtner et al., Arch. Dermatol. 141: 217-224, 2005, which are incorporated herein by reference. The at least one sensor may also detect skin conductivity at the insertion-target region. Following a determination of skin subsurface structure by the sensor, the control circuitry is operable to output moveable needle targeting instructions to control movement of the moveable needle into a target location in the skin subsurface structure of the subject in response to one or more signals from the at least one sensor indicating the structure of the skin subsurface. The control circuitry is operably coupled to the at least one sensor operable to output moveable needle targeting instructions that determine the desired position and depth of the needle in the skin subsurface structure.

### Sensors and Control Circuitry Operable to Detect an Insertion Depth of the Movable Needle in Skin Structure

A needle insertion system includes control circuitry operably coupled to at least one second sensor. The at least one second sensor is operable to detect an insertion depth of the movable needle. The at least one second sensor is operable to provide one or more signals related to the insertion depth to the control circuitry, for example, through measurement of impedance. The at least one second sensor may include a first electrode, e.g., located at a distal end of the needle, and a second electrode, e.g., located at a distal end of the needle insertion system. The at least one second sensor may output an electronic signal associated with an impedance measurement between the first electrode and the second electrode. The control circuitry is operable to output movable needle targeting instructions to control movement of the moveable needle into the skin in response to the one or more impedance measurement signals that correlate to the insertion depth. The at least one second sensor is operable to detect an insertion depth of the movable needle relative to at least one skin subsurface structure. The control circuitry in response to signals from the at least one sensor and the at least one second sensor can target the needle to a prescribed depth into the target tissue at the insertion/target region. The control circuitry may also provide electronic and/or automatic feedback associated with the validity and/or administration of an injection event based on the impedance between the first electrode and the second electrode. See, e.g., U.S. Patent Application 2009/0024112, which is incorporated herein by reference.

### Prophetic Exemplary Embodiments

### EXAMPLE 1

Device and Method for Auto-Injection of Medicaments at a Desired Depth Below the Surface of the Skin with a Needle Insertion System.

A needle insertion system includes automated medication injector constructed with a sensor to detect dermal and subdermal tissues. The sensor signals are analyzed by microcircuitry on the injector to determine the depth of injection and to signal an actuator to insert the injector needle to the desired depth and to expel the medication at a predetermined rate. Control circuitry on the automated injector is programmed to identify dermal and subdermal tissue layers based on sensor signals, and to determine the depth of injection based on the identified tissue layers thus controlling for injection at any site (arm, hip or scapular), individual patient variation (e.g., obese) and different specific medications. Data on the patient identity, the specific medication, the body site, tissue layer, depth, day and time of the injection are transmitted to a central computer and added to the patient's electronic health record.

The needle insertion system is constructed with an electronically actuated syringe and circuitry to control the movement of the needle to a desired tissue depth, and to control the rate of medication release from the needle. For example. the electromechanical device may incorporate a motor which drives a shaft attached to an injection needle at a constant rate for a specified time to insert the needle a specified depth into the skin. A second motor and shaft may drive a syringe plunger at a constant rate and for a specified time to deliver a specified dose of medication at a specified rate. An injection device may include a moveable needle and a plunger responsive to electronic signals. See e.g., U.S. Patent Application No. 2009/0024112 by Edwards et al. published on Jan. 22, 2009 which is incorporated herein by reference. Microcircuitry in the device contains stored data to identify an optimal dermal or subdermal tissue layer for injection of the medication being administered. The optimal rate of medication release is also stored in the microcircuitry of the device. A needle insertion system may include a moveable needle controlled by microcircuitry and a drive unit to inject a fluid at a predetermined flow rate. See e.g., U.S. Pat. No. 7,740,612 issued to Hochman on Jun. 22, 2010, which is incorporated herein by reference. The automated injection device includes a sensor to image the tissue at the injection site and determine injection depth.

The needle insertion system incorporates an ultrasound (US) transducer to detect the depth of dermal and subdermal layers beneath the skin surface. The transducer emits US waves into the tissue at the target injection site and senses echoes of the US waves reflected by dermal and subdermal tissues. Control circuitry on the autoinjector records and stores the times for return echoes at the injection site, and the depth of tissue layers beneath the skin is calculated based on the echo times. The distances of dermal layers and their interfaces beneath the skin are determined. For example, the depth below the skin surface of the interface between the reticular dermis and the papillary dermis may be determined. See e.g., U.S. Pat. No. 6,135,994 issued to Chernoff on Oct. 24, 2000 and U.S. Pat. No. 7,722,535 issued to Randlov et al. on May 25, 2010 which are incorporated herein by reference. Ultrasound transducers producing high frequency (approximately 20-50 MHz), and midrange frequencies (in the range of 7.5 to 10 MHz) are available from Cortex Technology, Hadsund, Denmark and Acuson, Mountain View, Calif., respectively. B-mode signal processing is used to produce “images” representing the reflections of US waves from epidermal and dermal tissue layers. A needle insertion system utilizes ultrasound in the analysis of skin layers, subcutaneous tissues, and tissue interfaces. See e.g., Schmid-Wendtner et al., Arch. Dermatol. 141: 217-224, 2005, which is incorporated herein by reference. In addition the automated injector may include an ultrafast ultrasonic scanner which generates a shear wave and images the tissues during wave propagation at a very high frame rate (e.g., up to 6000 images/s). The shear wave is distorted by tissues with differing elasticity and an elasticity map is calculated which indicates the position of tissues with different elasticity. For example ultrasound shear wave imaging is used to map heterogeneity in human breast tissue and in skin. See e.g., Bercoff et al., IEEE Transactions on Ultrasonics, Ferroelectrics, and Frequency Control 51: 396-409, 2004; and Wells et al., Journal Royal Soc. Interface 8: 1521-1549, 2011 which are incorporated herein by reference.

Alternatively the needle insertion system may incorporate an optical coherence tomography (OCT) imaging system to image dermal tissues and determine the injection target site and the injection depth method for inserting a movable needle of a needle insertion system into a vertebrate subject may utilize in-depth imaging of skin using OCT. See e.g., Wen et al., J. Biomedical Optics 17(6), 066022 (June 2012) available online at: www.SPIEDigitalLibrary.org/jbo which is incorporated herein by reference. An OCT imaging system provides good resolution (e.g., 12 μm axial resolution and 25 μm lateral resolution) and penetration (e.g., 12 mm) for imaging tissues. See e.g., ThorLabs, Newton, N.J.; “ThorLab- MEMS-VCSEL Swept Source OCT InfoSheet” available online at:
http://www.thorlabs.com/newgrouppage9.cfm?objectgroup_id=6473&pn=OCS1310V1 which is incorporated herein by reference. The OCT imaging system incorporates a 3D scanning probe and an integrated video camera; the system can image depth, planes and volumes of tissues in vivo, and map injection sites.

The The needle insertion system also has an impedance measurement system to measure and report the depth of needle insertion. For example the injection device has two electrodes: one at the tip of the needle and a second on the tip of the syringe barrel to allow measurement of impedance of the tissue between the electrodes. Upon injection of a medicament an electrical current is delivered between the electrodes and circuitry on the device measures the impedance between the electrodes. The measured impedance will reflect the depth of needle insertion and the character of tissues surrounding the needle, thus providing feedback that the automated injection is at the intended depth in the intended skin layer. A needle insertion system may include a device to measure impedance at an injection site. See e.g., U.S. Patent Application No. 2009/0024112 Ibid. Moreover, the impedance measurement may be made prior to releasing, i.e., injecting, medicament from the device to confirm the needle is inserted at the intended depth, and is targeted to the intended skin layer before injecting medicament.

Injection site locations and images may be stored in the circuitry on the device or relayed wirelessly to a central computer containing electronic health records. Data on the injection site location, depth of injection, date, time, patient, and medication are stored in memory on the automated injector system and/or on a central computer.

### EXAMPLE 2

### Device and Method for Influenza Vaccination in Epidermis and Dermis with a Needle Insertion System.

A needle insertion system includes an automated injection device with a high frequency ultrasound (US) transponder that is used to inject an influenza vaccine intradermally. Reduced doses of influenza vaccine may be sufficient to induce immunity when they are administered intradermally. See e.g., Hickling et al., Bull. World Health Organ. 89: 221-226, 2011 which is incorporated herein by reference. The needle insertion system detects epidermal and dermal layers and their depths beneath the skin and automatically injects influenza vaccine in the preferred skin sublayer.

The needle insertion system uses US sensors to locate epidermal and dermal layers and a needle deployment system is used to deliver an influenza vaccine to the epidermis and dermis. The automated injection device is used to accurately deliver vaccines and promote the activation of Langerhans cells and dendritic cells which reside in the epidermis and dermis respectively. See e.g. Poulin et al., J. Exp. Med. 204: 3119-3131, 2007 which is incorporated herein by reference. For example, injection of influenza vaccine in the epidermis may lead to strong immune responses with reduced amounts of vaccine. See e.g., Song et al., Clin. Exp. Vaccine Res. 2: 115-119, 2013 which is incorporated herein by reference. Reduced doses of a flu vaccine are delivered to the epidermis, e.g., the stratum spinosum, and to the dermis, e.g., papillary dermis, using the automated injection device to locate the skin sublayers and to determine the depth of the injections. For example the device may have a high frequency ultrasound (US) system, e.g., a 20 MHz system. See, e.g., Cortex Technology, Hadsund, Denmark; see the DermaScan® C USB Brochure available online at http://www.cortex.dk/skin-analysis-products/dermascan-ultrasound.html which is included herein by reference. A high frequency ultrasound (US) system, e.g., a 20 MHz system can penetrate to approximately 14 mm and provide axial resolution of 60 μm and lateral resolution of 150 μm to measure the depth of skin tissue layers, e.g., epidermis, papillary dermis, and reticular dermis. Reflected US waves (echoes) are detected as electrical signals which are processed to images corresponding to the skin topology. Image analysis circuitry and programming identify and measure the depth of the epidermal and dermal layers beneath the skin surface (programs for analyzing and measuring US images of skin are available from Cortex Technology Hadsund, Denmark (see e.g., DermaScan® Brochure, Ibid.). For example, the border between the epidermis and the papillary dermis may give rise to US echoes defining a boundary near the stratum spinosum approximately 0.20 mm below the skin surface in the deltoid area, and US echoes from the papillary/reticular dermal boundary may define a border approximately 1.2 mm beneath the skin surface. See e.g., Laurent et al., Vaccine 25: 6423-6430, 2007 which is incorporated herein by reference. To target delivery of an influenza vaccine to antigen presenting cells in the skin, e.g., Langerhans cells, which reside near the epidermis/dermis boundary, the injection device detects the epidermis/dermis boundary at approximately 0.20 mm depth and inserts the injection needle to 0.20 mm and injects a predetermined dose of flu vaccine, e.g., approximately 100 μL. To target dendritic cells which reside in the papillary dermis the device detects the papillary dermis by characteristic echoes and inserts the injection needle into the papillary dermis, approximately 1.00 mm beneath the skin surface, and delivers 100 μl influenza vaccine. See e.g., Laurent et al., Ibid. Delivery at two dermal layers, i.e., two depths in the skin may be done sequentially at one site on the skin or at two injection sites. The device is preprogrammed to select the skin layers or boundaries for injection and the dose of vaccine required. Once the device is programmed it determines the injection depth for each patient and each injection site using US imaging and releases the predetermined dose using automated activators for needle deployment and vaccine release (injection).

The needle insertion system is constructed with an electronically actuated syringe and circuitry to insert the needle to a desired depth beneath the skin, and to control the rate and dose of medication release from the needle. For example the electronically actuated syringe may be programmed to deliver 0.1 mL of a trivalent flu vaccine at the boundary of the epidermal and dermal layers, at a depth determined by the US system on the device. The electronically actuated syringe needle is deployed at the determined depth after the automated injection device is placed on the skin. See e.g., U.S. Pat. No. 8,308,741 and U.S. Pat. No. 6,547,755 which are incorporated herein by reference. For example a micro linear actuator with a range of 25.4 mm and accuracy of 15 μm is available from Zaber Technologies Inc., Vancouver, B.C., Canada (see e.g. Micro Linear Actuator Spec Sheet available online at http:/www.zaber.com/products/product_group.php?group=T-NA08A25-SV2 which is incorporated herein by reference). Electromechanical actuators with integrated controllers are also used to drive syringe plungers and deliver microliter volumes of influenza vaccine (e.g., 50-200 μL). Automated needle insertion at a preferred depth (dermal layer) for immunization combined with automated accurate delivery of reduced volumes of vaccine results in efficient vaccination independent of individual variation in skin thickness and operator error associated with intradermal injections. The needle insertion system wirelessly transmits data on the vaccination to a central computer: the date, time, patient, vaccine ID, lot no., site of the injection (e.g., left arm) needle insertion depth, skin sublayer and dose (e.g., volume) are reported.

Each recited range includes all combinations and sub-combinations of ranges, as well as specific numerals contained therein.

All publications and patent applications cited in this specification are herein incorporated by reference to the extent not inconsistent with the description herein and for all purposes as if each individual publication or patent application were specifically and individually indicated to be incorporated by reference for all purposes.

Those having ordinary skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having ordinary skill in the art will recognize that there are various vehicles by which processes and/or systems and/or other technologies disclosed herein can be effected (e.g., hardware, software, and/or firmware), and that the preferred vehicle will vary with the context in which the processes and/or systems and/or other technologies are deployed. For example, if a surgeon determines that speed and accuracy are paramount, the surgeon may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies disclosed herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those having ordinary skill in the art will recognize that optical aspects of implementations will typically employ optically-oriented hardware, software, and or firmware.

In a general sense the various aspects disclosed herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices disclosed herein, or a microdigital processing unit configured by a computer program which at least partially carries out processes and/or devices disclosed herein), electrical circuitry forming a memory device (e.g., forms of random access memory), and/or electrical circuitry forming a communications device (e.g., a modem, communications switch, or optical-electrical equipment). The subject matter disclosed herein may be implemented in an analog or digital fashion or some combination thereof.

At least a portion of the devices and/or processes described herein can be integrated into a data processing system. A data processing system generally includes one or more of a system unit housing, a video display device, memory such as volatile or non-volatile memory, processors such as microprocessors or digital signal processors, computational entities such as operating systems, drivers, graphical user interfaces, and applications programs, one or more interaction devices (e.g., a touch pad, a touch screen, an antenna, etc.), and/or control systems including feedback loops and control motors (e.g., feedback for sensing position and/or velocity; control motors for moving and/or adjusting components and/or quantities). A data processing system may be implemented utilizing suitable commercially available components, such as those typically found in data computing/communication and/or network computing/communication systems.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link (e.g., transmitter, receiver, transmission logic, reception logic, etc.), etc.).

The herein described components (e.g., steps), devices, and objects and the description accompanying them are used as examples for the sake of conceptual clarity and that various configuration modifications using the disclosure provided herein are within the skill of those in the art. Consequently, as used herein, the specific examples set forth and the accompanying description are intended to be representative of their more general classes. In general, use of any specific example herein is also intended to be representative of its class, and the non-inclusion of such specific components (e.g., steps), devices, and objects herein should not be taken as indicating that limitation is desired.

With respect to the use of substantially any plural or singular terms herein, the reader can translate from the plural to the singular or from the singular to the plural as is appropriate to the context or application. The various singular/plural permutations are not expressly set forth herein for sake of clarity.

The herein described subject matter sometimes illustrates different components contained within, or connected with, different other components. It is to be understood that such depicted architectures are merely examples, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected,” or “operably coupled,” to each other to achieve the desired functionality, and any two components capable of being so associated can also be viewed as being “operably couplable,” to each other to achieve the desired functionality. Specific examples of operably couplable include but are not limited to physically mateable or physically interacting components or wirelessly interactable or wirelessly interacting components or logically interacting or logically interactable components.

While particular aspects of the present subject matter described herein have been shown and described, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an”; the same holds true for the use of definite articles used to introduce claim recitations. In addition, even if a specific number of an introduced claim recitation is explicitly recited, such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, or A, B, and C together, etc.). Virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A needle insertion system comprising:
a moveable hollow needle having a distal tip configured to be transdermally inserted into a vertebrate subject at an insertion-target region;
at least one sensor operable to detect skin structure at a position in proximity to the distal tip of the moveable needle in the insertion-target region; and
control circuitry operably coupled to the at least one sensor and configured to receive information therefrom indicating the skin structure at the insertion-target region in proximity to the distal tip, and the control circuitry configured to output moveable needle targeting instructions to control movement of the moveable needle into the skin in response to one or more signals from the at least one sensor detecting the skin structure in proximity to the distal tip.

2. The system of claim 1, wherein the at least one sensor is operable to detect the skin thickness of the vertebrate subject utilizing ultrasound longitudinal waves or ultrasound shear waves.

3. The system of claim 1, wherein the at least one sensor is operable to detect the skin thickness of the vertebrate subject utilizing optical coherence tomography.

4. The system of claim 1, wherein the at least one sensor is operable to detect skin conductivity.

5. The system of claim 1, wherein the skin structure includes skin subsurface structure.

6. The system of claim 5, wherein the skin subsurface structure includes at least one of size, location, or thickness of dermis, epidermis, hypodermis, papillary region of the dermis, reticular region of the dermis, boundaries between the epidermis and the dermis, boundaries between the dermis and the hypodermis, and boundaries between the reticular and papillary regions.

7. The system of claim 5, wherein the control circuitry is operable to control insertion depth of the distal tip of the needle relative to the skin subsurface structure.

8. The system of claim 7, wherein the control circuitry is operable to control insertion depth of the distal tip of the needle to a specified depth within dermis.

9. The system of claim 8, wherein the specified depth is within a papillary region of the dermis.

10. The system of claim 8, wherein the specified depth is within a reticular region of the dermis.

11. The system of claim 8, wherein the specified depth is proximate to a boundary between a papillary region and a reticular region of the dermis.

12. The system of claim 7, wherein the control circuitry is operable to control insertion depth of the needle to stop prior to entering hypodermis.

13. The system of claim 7, wherein the control circuitry is operable to control insertion depth of the needle such that a fluid delivery opening at the distal tip of the needle is at a specified depth within dermis.

14. The system of claim 1, wherein the control circuitry operably coupled to the at least one sensor is configured to receive location information therefrom about the insertion-target region.

15. The system of claim 1, further comprising at least one second sensor operable to detect an insertion depth of the movable needle, and to provide one or more signals related to the insertion depth to the control circuitry.

16. The system of claim 15, where the control circuitry is operable to output movable needle targeting instructions to control movement of the moveable needle into the skin in response to the one or more signals related to the insertion depth.

17. The system of claim 15, wherein the at least one second sensor is operable to detect an insertion depth of the distal tip of the movable needle relative to at least one skin subsurface structure.

18. A method for inserting a movable hollow needle of a needle insertion system into a vertebrate subject, comprising:
locating an insertion-target region on the vertebrate subject with at least one sensor operable to detect skin structure at a position in proximity to the distal tip of the moveable needle in the insertion-target region;
outputting location information from the at least one sensor to control circuitry including the location of the insertion-target region and skin structure information in proximity to the distal tip; and
automatically moving the needle to a defined depth into skin at the insertion-target region of the vertebrate subject in response to needle-targeting instructions output from the control circuitry.

19. The method of claim 18, wherein automatically moving the needle to the defined depth into the skin in response to the needle-targeting instructions output from the control circuitry occurs without human intervention and after the needle is in operational range relative to the vertebrate subject.

20. The method of claim 18, wherein automatically moving the needle to the defined depth into the skin in response to the needle-targeting instructions output from the control circuitry includes automatically moving the needle to the defined depth into the skin with an actuator that receives the needle-targeting instructions.

21. The method of claim 18, wherein the skin structure includes skin subsurface structure.

22. The method of claim 21, wherein the skin subsurface structure includes at least one of size, location, or thickness of dermis, epidermis, hypodermis, papillary region of the dermis, reticular region of the dermis, boundaries between the epidermis and the dermis, boundaries between the dermis and the hypodermis, and boundaries between the reticular and papillary regions.

23. The method of claim 21, comprising controlling with the control circuitry insertion depth of the distal tip of the needle relative to the skin subsurface structure.

24. The method of claim 23, comprising controlling with the control circuitry insertion depth of the distal tip of the needle to a specified depth within dermis.

25. The method of claim 24, wherein the specified depth is within a papillary region of the dermis.

26. The method of claim 24, wherein the specified depth is within a reticular region of the dermis.

27. The method of claim 24, wherein the specified depth is proximate to a boundary between a papillary region and a reticular region of the dermis.

28. The method of claim 23, wherein the control circuitry is configured to control insertion depth of the needle to stop prior to entering hypodermis.

29. The method of claim 23, wherein the control circuitry is configured to control insertion depth of the needle such that a fluid delivery opening at the distal tip of the needle is at a specified depth within dermis.

30. The method of claim 18, comprising automatically moving the needle to the defined depth at one or more of an epidermal layer, dermal layer, and hypodermal layer of the skin.

31. The method of claim 18, comprising outputting location information from the at least one sensor to the control circuitry including the skin structure information of the size or thickness of dermis, epidermis, hypodermis, papillary region of the dermis, reticular region of the dermis, boundaries between the epidermis and the dermis, boundaries between the dermis and the hypodermis, and boundaries between the reticular region and the papillary region.

32. The method of claim 18, comprising locating the insertion-target region on the vertebrate subject with the at least one sensor operable to detect the skin thickness of the vertebrate subject utilizing ultrasound longitudinal waves or ultrasound shear waves.

33. The method of claim 18, comprising locating the insertion-target region on the vertebrate subject with the at least one sensor operable to detect the skin thickness of the vertebrate subject utilizing optical coherence tomography.

34. The method of claim 18, comprising locating the insertion-target region on the vertebrate subject with the at least one sensor operable to detect skin conductivity.

35. The method of claim 18, comprising outputting location information including an insertion depth of the movable needle from at least one second sensor to the control circuitry.

36. The method of claim 35, comprising outputting movable needle targeting instructions from the control circuitry to control movement of the moveable needle into the skin in response to the one or more signals from the least one second sensor related to the insertion depth.

37. The method of claim 35, comprising detecting with the least one second sensor an insertion depth of the distal tip of the movable needle relative to at least one skin subsurface structure.
